(19) United States
(12) Patent Application Publication
(10) Pub. No.: US 2025/0235433 A1
(43) Pub. Date: Jul. 24, 2025

(54) METHODS OF TREATING AND DIAGNOSING FATTY LIVER DISEASE

(71) Applicants: CEDARS-SINAI MEDICAL CENTER, Los Angeles, CA (US); NATIONAL AND KAPODISTRIAN UNIVERSITY OF ATHENS

(72) Inventors: Konstantinos Triantafyllou, Alimos (GR); Mark Pimentel, Los Angeles, CA (US); Ruchi Mathur, Los Angeles, CA (US); Ali Rezaie, Beverly Hills, CA (US); Gabriela Guimaraes Sousa Leite, Porter Ranch, CA (US)

(73) Assignees: CEDARS-SINAI MEDICAL CENTER, Los Angeles, CA (US); NATIONAL AND KAPODISTRIAN UNIVERSITY OF ATHENS, (omitted)

(21) Appl. No.: 18/855,498

(22) PCT Filed: Apr. 18, 2023
(86) PCT No.: PCT/US2023/065905

§ 371 Date: Oct. 9, 2024

Related U.S. Application Data
(60) Provisional application No. 63/332,573, filed on Apr. 19, 2022.

(51) Int. Cl. A61K 31/43; A61K 35/74; C12Q 1/689; G01N 33/84

(52) U.S. Cl. CPC A61K 31/43; A61K 35/74; C12Q 1/689; G01N 33/84; C12Q 2600/156; G01N 2800/085

(57) ABSTRACT

Described herein are methods of treating and detecting nonalcoholic fatty liver disease (NAFLD) including nonalcoholic fatty liver (NAFL) and non-alcoholic steatohepatitis (NASH).

### CROSS-REFERENCE TO RELATED APPLICATIONS

This application includes a claim of priority under 35 U.S.C. § 119 (e) to U.S. provisional patent application No. 63/332,573, filed Apr. 19, 2022, the entirety of which is hereby incorporated by reference.

### FIELD OF INVENTION

This invention relates to the treatment and detection of fatty liver disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Nonalcoholic fatty liver disease (NAFLD) refers to a range of liver conditions affecting people who drink little to no alcohol. Nonalcoholic fatty liver (NAFL) and nonalcoholic steatohepatitis (NASH) are types of NAFLD. A main characteristic of NAFLD is too much fat stored in liver cells. NAFLD is increasingly common around the world. In the United States, it is the most common form of chronic liver disease, affecting about one-quarter of the population.

Some individuals with NAFLD can develop nonalcoholic steatohepatitis (NASH), an aggressive form of fatty liver disease, which is marked by liver inflammation and may progress to cirrhosis and liver failure.

Diagnostic tests include various blood tests and imaging tests but are not specific for the detection of NAFLD. Current treatment options include weight loss though a healthy diet and exercise. Currently, no drug treatment has been approved by the Food and Drug Administration (FDA).

As such, there remains a need in the art for methods of diagnosing and treating NAFLD.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a method of treating nonalcoholic fatty liver disease (NAFLD) in a subject in need thereof, comprising administering to the subject an agent that inhibits or eradicates actinomyces.

In various embodiments, the NAFLD can be nonalcoholic fatty liver (NAFL). In various embodiments, the NAFLD can be non-alcoholic steatohepatitis (NASH).

In various embodiments, the agent that inhibits, redistributes, reduces, or eradicates actinomyces can comprise an antibiotic. For example, the antibiotic can be Penicillin G. In other examples, the antibiotic can be rifaximin, amoxicillin, ceftriaxone, clarithromycin, clindamycin, doxycycline, erythromycin, linezolid, meropenem, penicillin G, tazobactam, tetracycline or combinations thereof.

In various embodiments, the agent can comprise prevotella.

In various embodiments, the agent can be released into the gastrointestinal system of the subject. In various embodiments, the agent can be released into duodenum of the subject.

Various embodiments provide for a method of diagnosing nonalcoholic fatty liver disease (NAFLD) in a subject in need thereof, comprising: measuring a level of actinomyces in the gastrointestinal system of the subject; and determining the presence of NALFD when a level of actinomyces is higher than a control level.

In various embodiments, NAFLD can be nonalcoholic fatty liver (NAFL) and the level of actinomyces is higher than a control level for NALF. In various embodiments, NAFLD can be non-alcoholic steatohepatitis (NASH), and the level of actinomyces is higher than a control level for NASH.

In various embodiments, the level of actinomyces in the gastrointestinal system can be measured from a biological sample selected from saliva, small intestinal aspirate, or stomach aspirate.

Various embodiments of the invention provide for a method of diagnosing nonalcoholic fatty liver disease (NAFLD) in a subject in need thereof, comprising: measuring a level of sulfur granules in the gastrointestinal system of the subject; and determining the presence of NALFD when a level of sulfur granules is higher than a control level.

In various embodiments, the NAFLD can be nonalcoholic fatty liver (NAFL) and the level of actinomyces is higher than a control level for NALF. In various embodiments, the NAFLD can be non-alcoholic steatohepatitis (NASH), and the level of actinomyces is higher than a control level for NASH.

In various embodiments, the level of sulfur granules can be measured from a biological sample selected from saliva, small intestinal aspirate, or stomach aspirate. In various embodiments, small intestinal aspirate can be duodenal aspirate.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts Sunburst graphical representation of the small bowel microbiome profile (duodenum microbiome), at different taxonomic level, of subjects without NAFLD (Controls) and subjects with NAFLD, and shows that increased actinobacteria still holds in patients with NAFLD only.

FIG. 2 depicts small bowel microbial diversity (alpha diversity) of subjects without NAFLD (controls—in red) and subjects with NAFLD (in green). These figures show no differences in overall bacterial richness and evenness between groups despite the increased relative abundance of the genus Actinomyces.

FIGS. 3A-3B depict the graphical representation of Partial Least Squares Discriminant Analysis (PL-SDA) of the small bowel microbiome profile of subjects without NAFLD (red (left)) and with NAFLD (green (right)) after supervised dimensionality-reduction for feature selection. This analysis discriminates the most important differences between group.

FIG. 3C shows Pcol distance and PLS-DA analysis between control and NALFD.

FIG. 4A shows a heatmap of the most important small bowel microbial differences between groups after performing PL-SDA analysis (FIGS. 3A and 3B). It is possible to see a separation of subjects with NAFLD (green) from those without NAFLD (red). This heatmap shows that is possible to distinguish subjects with NAFLD from controls based on the microbiome profile.

FIG. 4B shows the most important bacterial genera that are different between groups, including an increased relative abundance of the genus Actinomyces in NAFLD (second row) and decreased relative abundance of Prevotella (first row). Profile based on 16S IRNA sequencing. F16 genus (TM7 phylum).

FIG. 5A depicts the graphical representation of Partial Least Squares Discriminant Analysis (PL-SDA) of the small bowel microbial metabolic pathways prediction of subjects without NAFLD (red) and with NAFLD (green) after supervised dimensionality-reduction for feature selection. This analysis discriminates the most important differences between group.

FIG. 5B shows the most important microbial metabolic pathways that are different between groups after PL-SDA (FIG. 5A), including an enrichment of the sulfur-relay system in subjects with NAFLD.

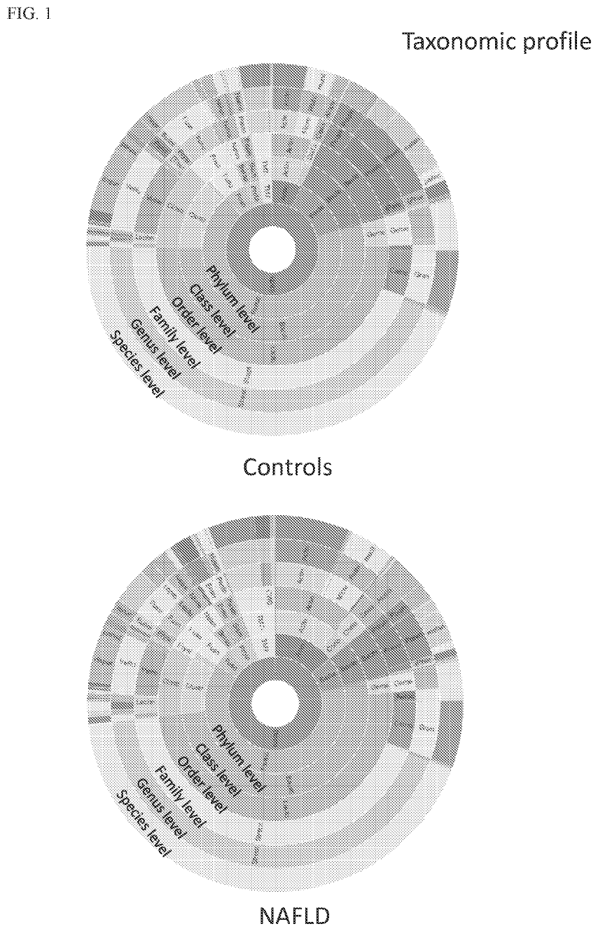

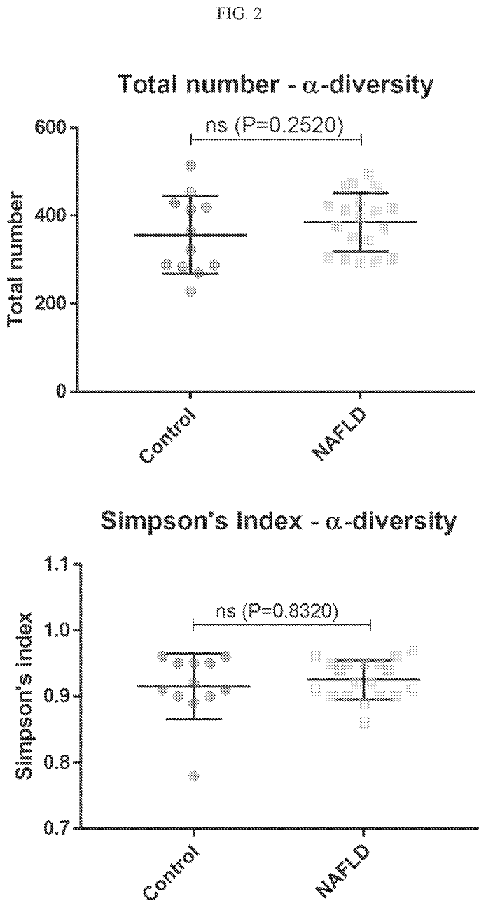

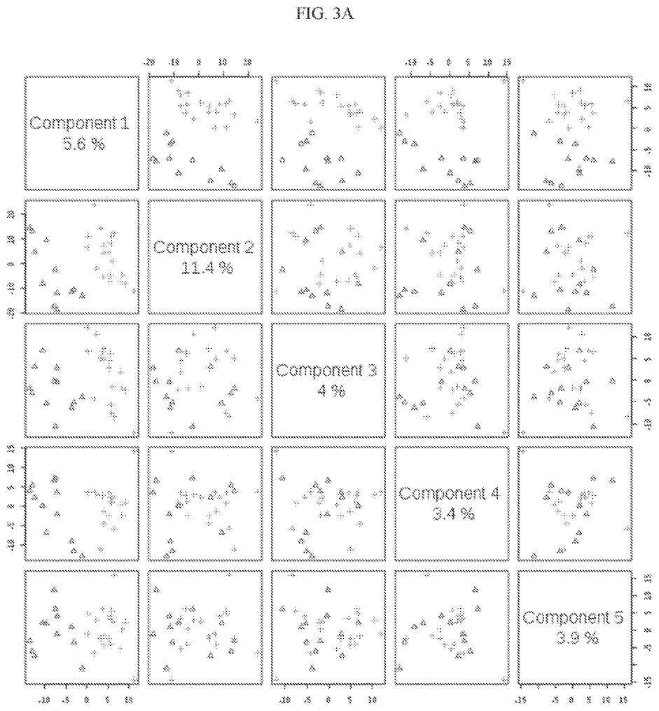

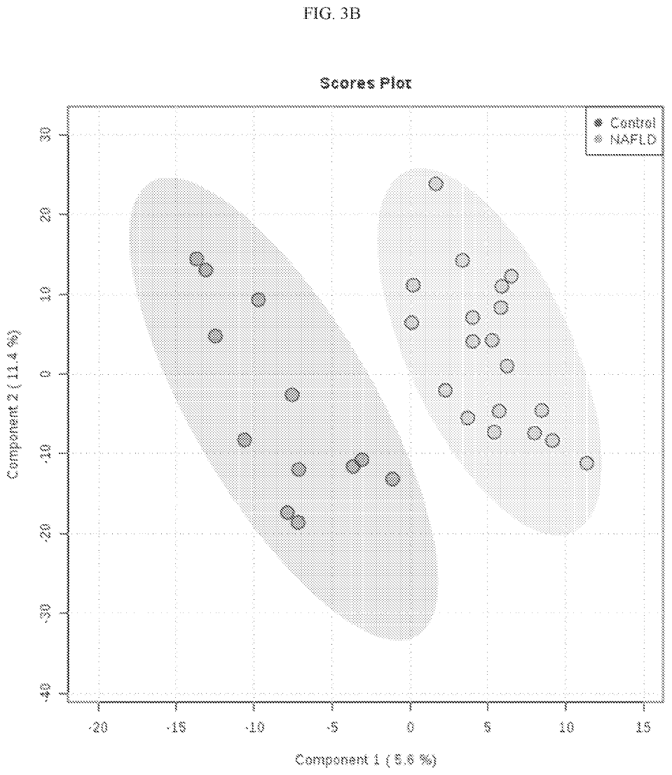

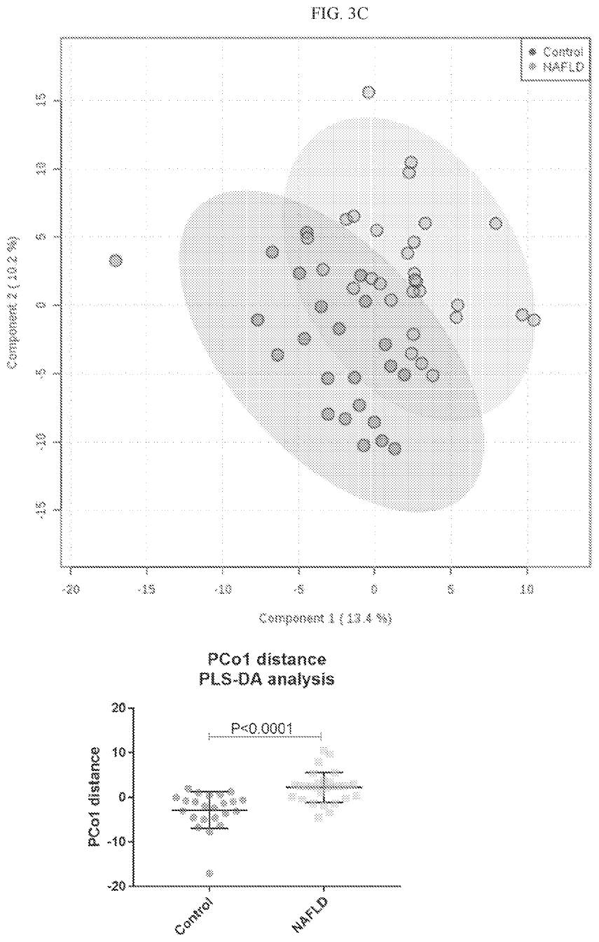

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein “substantial” when used in connection with a referenced numeric indication means an amount of at least 60% of the referenced numeric indication, unless otherwise specifically provided for herein. In various embodiments, the term “substantial” when used in connection with a referenced numeric indication can mean an amount of at least 65%, 70%, 75%, 80%, 85%, 90%, 95%, 96%, 97%, 98%, or 99% of the referenced numeric indication, if specifically provided for in the claims.

As used herein, the terms “treat,” “treatment,” “treating,” or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented. Non-limiting examples of treatments or therapeutic treatments include at least one selected from pharmacological therapies, biological therapies, interventional surgical treatments, and combinations thereof.

Examples of biological samples referred to herein include but are not limited to body fluids, stool, intestinal fluids or aspirate including duodenal fluids or aspirate, stomach fluids or aspirate, cerebral spinal fluid (CSF), urine, sweat, saliva, tears, pulmonary secretions, breast aspirate, prostate fluid, seminal fluid, cervical scraping, amniotic fluid, intraocular fluid, mucous, and moisture in breath. In various embodiments of the invention, the biological sample may be stool, intestinal fluid or aspirate including duodenum fluids or aspirate, stomach fluid, or aspirate, saliva. In particular embodiments, the biological sample may be intestinal aspirate, such as duodenal fluids or aspirate. In particular embodiments, the biological sample may be saliva. In particular embodiments, the biological sample may be stomach aspirate.

Through 16S rRNA sequencing analysis of biological samples such as duodenal aspirates, the inventors have found that all NAFLD patients have Actinomyces and particularly Actinomyces massiliensis, present whereas control patients have less or do not have any Actinomyces present. The duodenal microbiome of NAFLD patients is different at phylum and order level compared to that of control patients. A deeper assessment of the small bowel microbiome in this population lead to findings regarding the development of NAFLD. Various embodiments of the present invention are based, in part, on these findings.

Various embodiments of the present invention provide for a method of treating nonalcoholic fatty liver disease (NAFLD) in a subject in need thereof, comprising administering to the subject an agent that inhibits, redistributes, reduces, or eradicates actinomyces. In various embodiments, NAFLD is nonalcoholic fatty liver (NAFL). In various embodiments, NAFLD is non-alcoholic steatohepatitis (NASH).

Various embodiments of the present invention provide for a method of alleviating a symptom of nonalcoholic fatty liver disease (NAFLD) in a subject in need thereof, comprising administering to the subject an agent that inhibits, redistributes, reduces, or eradicates actinomyces. In various embodiments, NAFLD is nonalcoholic fatty liver (NAFL). In various embodiments, NAFLD is non-alcoholic steatohepatitis (NASH).

Various embodiments of the present invention provide for a method of inhibiting redistributing, reducing, or eradicating actinomyces to a subject having nonalcoholic fatty liver disease (NAFLD) or a symptom of NALFD, comprising administering to the subject the agent that inhibits, redistributes, reduces, or eradicates actinomyces. Particularly, Actinomyces massiliensis is inhibited, redistributed, reduced or eradicated.

In various embodiments, NAFLD is nonalcoholic fatty liver (NAFL). In various embodiments, NAFLD is non-alcoholic steatohepatitis (NASH).

Examples of symptoms of NALFD include but are not limited to fatigue, pain or discomfort in the upper right abdomen. Examples of symptoms of NASH include but are not limited to ascites, enlarged blood vessels (e.g., beneath the skin surface), enlarged spleen, red palms, and jaundice.

In various embodiments, the agent that inhibits, redistributes, reduces, or eradicates actinomyces comprises an antibiotic. In various embodiments, the antibiotic is Penicillin G. Additional examples of antibiotics that can be used include but are not limited to rifaximin, amoxicillin, ceftriaxone, clarithromycin, clindamycin, doxycycline, erythromycin, linezolid, meropenem, penicillin G, tazobactam, tetracycline or combinations thereof.

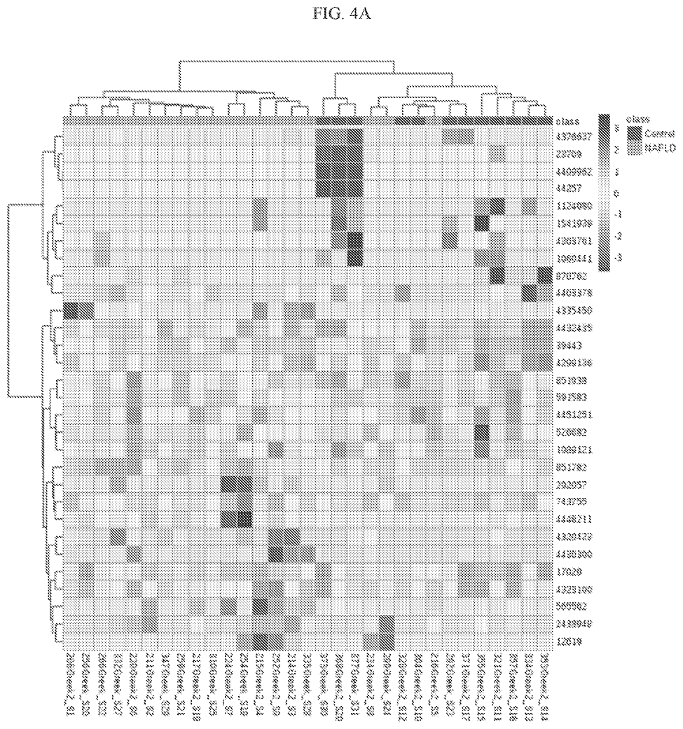

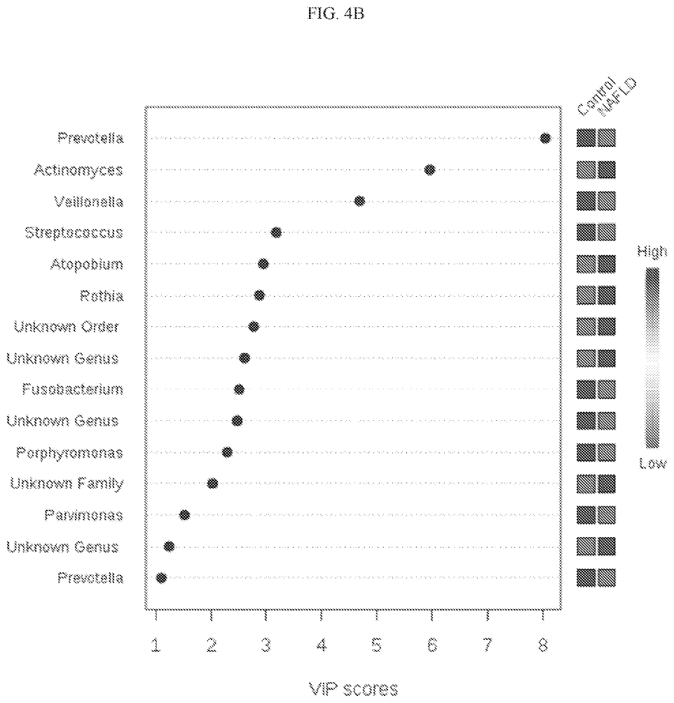

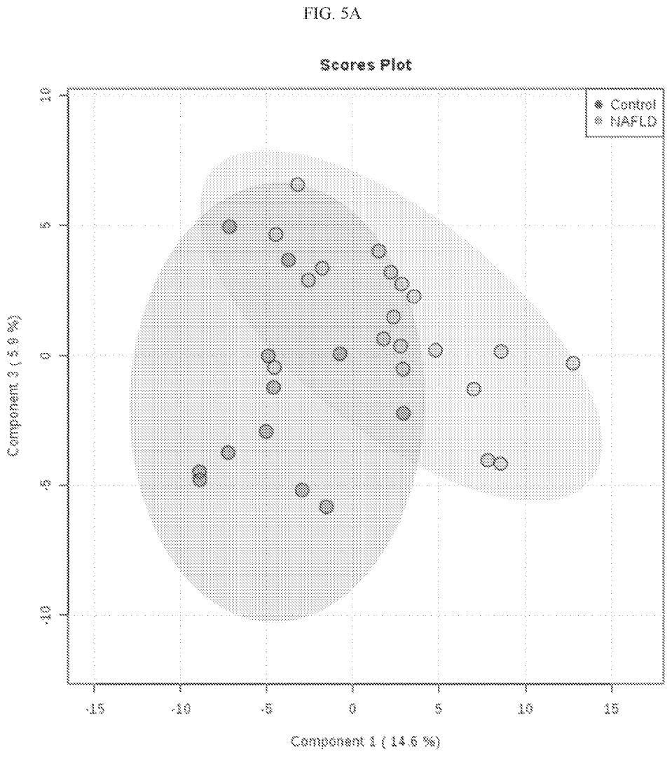

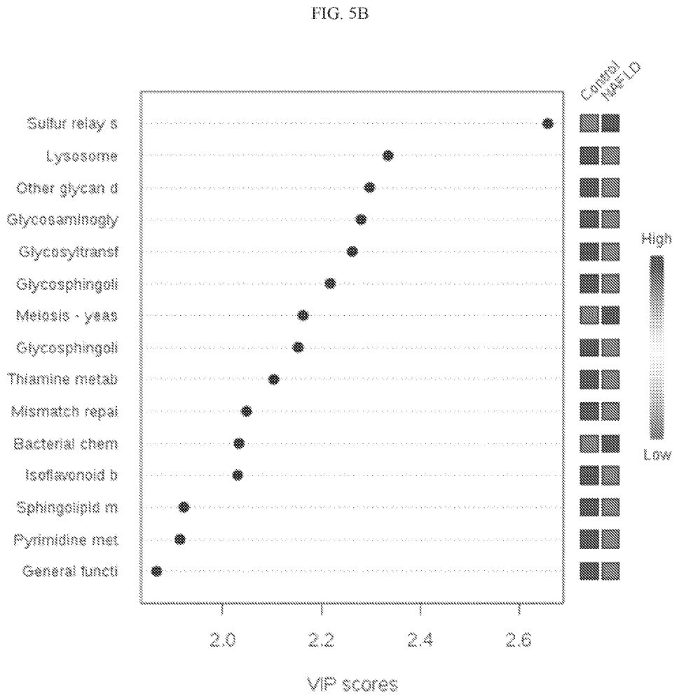

In various embodiments, the agent comprises Prevotella. While not wishing to be bound by any particular theory, the inventors believe that Prevotella inhibits the growth of actinomyces by outcompeting actinomyces or eradicates actinomyces by killing actinomyces. For example, FIG. 4B shows that while the relative abundance of Actinomyces increased in subjects with NAFLD, the relative abundance of Prevotella decreased (top 1 difference). This shows a competitive relationship between these two microorganisms; thus, Prevotella can be used as therapeutics.

In various embodiments, the Prevotella is Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella maculosa, Prevotella marshii, Prevotella melaninogenica, Prevotella micans, Prevotella multiformis, Prevotella oralis, Prevotella oris, Prevotella oulorum, Prevotella pallens, Prevotella salivae, Prevotella stercorea, Prevotella timonensis, Prevotella veroralis or combinations thereof. Particularly, Prevotella histicola, Prevotella oralis, Prevotella veroralis, Prevotella buccae, or combinations thereof can be beneficial. In particular embodiments, the Prevotella is Prevotella histicola.

Dosages of Prevotella that are provided or administered can be about 109 to 1010 CFU per dose. Additional examples include but are not limited to 108, 109, 1010, 58, 59, 510 CFR per dose.

In various embodiments, the agent is released into the gastrointestinal system of the subject. For example, the agent can be released into the mouth, pharynx (throat), esophagus, stomach, small intestine, large intestine, rectum, or anus. In various embodiments, the agent is released into duodenum of the subject. For example, the agent can be enteric-coated such that all or a substantial amount of the agent passes through the stomach and is released into the small intestine; for example, into the duodenum. In various embodiments, the agent is released into the mouth of the subject.

In various embodiments, the method further comprises measuring a level of actinomyces in the gastrointestinal system of the subject, and determining the presence of NALFD when a level of actinomyces is higher than a control level prior to administering the agent that inhibits, redistributes, reduces, or eradicates actinomyces. In various embodiments, the biological sample is duodenal aspirate or saliva. In particular, levels of Actinomyces massiliensis is measured and compared to the control level of Actinomyces massiliensis.

In various embodiments, the method further comprises measuring a level of sulfur granules in the gastrointestinal system of the subject, and determining the presence of NALFD when a level of sulfur granules is higher than a control level prior to administering the agent that inhibits, redistributes, reduces, or eradicates actinomyces. In various embodiments, the biological sample is duodenal aspirate or saliva. Particularly, the biological sample is small intestinal aspirate (e.g., duodenal aspirate), or saliva.

In various embodiments, the subject has been detected to have a level of actinomyces in the gastrointestinal system that is higher than a control level. For example, the subject has a level of Actinomyces massiliensis in the GI system that is higher than its control level.

In various embodiments, the biological sample wherein the level of actinomyces is detected from is a duodenal aspirate or saliva. Particularly, from duodenal aspirate or saliva. In various embodiments, the NAFLD is nonalcoholic fatty liver (NAFL) and the level of actinomyces (e.g., Actinomyces massiliensis) is higher than a control level for NALF. In various embodiments, the NAFLD is non-alcoholic steatohepatitis (NASH), and the level of actinomyces (e.g., Actinomyces massiliensis) is higher than a control level for NASH.

In various embodiments, the subject has been detected to have a level of sulfur granules in the gastrointestinal system that is higher than a control level. In various embodiments, the biological sample wherein the level of sulfur granules is detected from is a duodenal aspirate or saliva. In various embodiments, the NAFLD is nonalcoholic fatty liver (NAFL) and the level of sulfur granules is higher than a control level for NALF. In various embodiments, the NAFLD is non-alcoholic steatohepatitis (NASH), and the level of sulfur granules is higher than a control level for NASH.

Various embodiments of the present invention provide for a method of diagnosing nonalcoholic fatty liver disease (NAFLD) in a subject in need thereof, comprising: measuring a level of actinomyces in the gastrointestinal system of the subject; determining the presence of NALFD when a level of actinomyces is higher than a control level. For example, when a level of Actinomyces massiliensis is higher than its control level. In various embodiments, the biological sample is a duodenal aspirate. In various embodiments, the biological sample wherein the level of actinomyces is detected from is a duodenal aspirate or saliva. In various embodiments, the NAFLD is nonalcoholic fatty liver (NAFL) and the level of actinomyces is higher than a control level for NALF. For example, when a level of Actinomyces massiliensis is higher than its control level for NALF. In various embodiments, the NAFLD is non-alcoholic steatohepatitis (NASH), and the level of actinomyces is higher than a control level for NASH. For example, when a level of Actinomyces massiliensis is higher than its control level for NASH.

In various embodiments, measuring a level of actinomyces (e.g., Actinomyces massiliensis), comprises using a technique selected from the group consisting of PCR, DNA sequencing to determine the presence of actinomyces DNA, culturing for the actinomyces, 16S IRNA sequencing, and combinations thereof. Examples of DNA sequencing include but are not limited to Sanger sequencing, shotgun sequencing, and high-throughput sequencing (e.g., next-generation “short-read” and third-generation “long-read” sequencing methods (e.g., single molecule real time (SMRT) sequencing, nanopore DNA sequencing).

Various embodiments of the present invention provide for a method of diagnosing nonalcoholic fatty liver disease (NAFLD) in a subject in need thereof, comprising: measuring a level of sulfur granules in the gastrointestinal system of the subject; determining the presence of NALFD when a level of sulfur granules is higher than a control level. In various embodiments, the biological sample is a duodenal aspirate. In various embodiments, the biological sample wherein the level of sulfur granules is detected from is a duodenal aspirate or saliva. In various embodiments, the NAFLD is nonalcoholic fatty liver (NAFL) and the level of sulfur granules is higher than a control level for NALF. In various embodiments, the NAFLD is non-alcoholic steatohepatitis (NASH), and the level of sulfur granules is higher than a control level for NASH.

Control levels in accordance with various embodiments of the invention described herein can be established from biological samples from a healthy subject. In other embodiments, the control levels can be established from biological samples from a population of subject having NAFLD. In other embodiments, the control levels can be established from biological samples from a population of subject having NASH. In other embodiments, the control levels can be established from biological samples from a population of subject having NAFL.

For example, when establishing a control level based on healthy subject, if the biological sample is intestinal aspirate, then the control level can be obtained from the intestinal aspirate of a healthy subject. In some embodiments, the population of healthy subjects can range from at least three healthy individuals to 25 healthy individuals, and even more than 50 healthy individuals. A healthy individual can be an individual without liver disease. Similarly, when the control level is based on subjects having NAFLD, NASH, or NALF, the population of each of those subjects can range from at least three individuals to 25 healthy individuals, and even more than 50 individuals.

As further examples, the control level of actinomyces (e.g., Actinomyces massiliensis) can be the average level of actinomyces from a number of control subjects, for example, 10-25 subjects, 10-50 subjects, 10-100 subjects, 25-50 subjects, 25-100 subjects, 25-150 subjects, 50-100 subjects, 50-150 subjects, 50-200 subjects, 50-250 subjects, 100-200 subjects, 100-250 subjects, 100-300 subjects, 100-400 subjects, 100-500 subjects, 100-1000 subjects, 500-750 subjects, 500-100 subjects, or 1000-2000 subjects, or more than 2000 subject. Typically, the control levels will also be matched to the type of sample. For example, if the biological sample to be tested is duodenal aspirate, the control level of actinomyces will be from duodenal aspirates from the control subject(s).

In various embodiments, the present invention provides pharmaceutical compositions including a pharmaceutically acceptable excipient along with a therapeutically effective amount of the agent that inhibits, reduces, redistributes or eradicates actinomyces. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals such as sodium, lithium, potassium, calcium, magnesium and the like, as well as nontoxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “pharmaceutically acceptable esters” refers to the relatively nontoxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs” are salts or prodrugs that are, within the scope of sound medical judgment, suitable for use in contact with the tissues of subject without undue toxicity, irritation, allergic response, and the like, commensurate with a reasonable benefit/risk ratio, and effective for their intended use.

The term “prodrug” refers to compounds that are rapidly transformed in vivo to yield the functionally active one or more peptides as disclosed herein or a mutant, variant, analog or derivative thereof. A thorough discussion is provided in T. Higachi and V. Stella, “Pro-drugs as Novel Delivery Systems,” Vol. 14 of the A. C. S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. A prodrug of the one or more peptides as disclosed herein or a mutant, variant, analog or derivative thereof can be designed to alter the metabolic stability or the transport characteristics of one or more peptides as disclosed herein or a mutant, variant, analog or derivative thereof, to mask side effects or toxicity, to improve the flavor of a compound or to alter other characteristics or properties of a compound. By virtue of knowledge of pharmacodynamic processes and drug metabolism in vivo, once a pharmaceutically active form of the one or more peptides as disclosed herein or a mutant, variant, analog or derivative thereof, those of skill in the pharmaceutical art generally can design prodrugs of the compound (see, e.g., Nogrady (1985) Medicinal Chemistry A Biochemical Approach, Oxford University Press, N. Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection.

Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### NAFLD Analysis

Control (N=24) vs. NAFLD (N=38)

Patients with histological, biochemical or radiological diagnosis of NAFLD underwent upper gastrointestinal endoscopy and duodenal juice (2 cc) was aspirated from the 3rd-4th part of duodenum in sterile traps. Patients without liver disease, undergoing gastroscopy due to GERD, comprised the control group. DNA was isolated using the MagAttract PowerSoil DNA Kit. Microbiota was analyzed by 16S rRNA metagenomic sequencing in MiSeq platform (Illumina). Operational Taxonomic Units (OTU) clustering and taxonomic analysis were performed with CLC Microbial Genomics Module v. 2.5. A Negative Binomial GLM model was used to obtain maximum likelihood estimates for an OTU's fold change between the two groups, and the Wald test was used to determine significance.

### Example 2

### First Report on the Differences Between Non-Alcoholic Fatty Liver Disease Patients and Healthy Subjects Based on the Duodenal Microbiome

We aimed to characterize the duodenal microbiome (DM) of NAFLD patients and compare it to that of healthy controls. Patients with histological, biochemical or radiological diagnosis of NAFLD underwent upper gastrointestinal endoscopy and duodenal juice (2 cc) was aspirated from the 3^{rd}-4^{th} part of duodenum in sterile traps. Patients without liver disease, undergoing gastroscopy due to GERD, comprised the control group. DNA was isolated using the MagAttract PowerSoil DNA Kit. Microbiota was analyzed by 16S rRNA metagenomic sequencing in MiSeq platform (Illumina). Operational Taxonomic Units (OTU) clustering and taxonomic analysis were performed with CLC Microbial Genomics Module v. 2.5. A Negative Binomial GLM model was used to obtain maximum likelihood estimates for an OTU's fold change between the two groups, and the Wald test was used to determine significance.

19 subjects with NAFLD (7 females; age 57.1±11.2 yrs) and 12 controls (7 females; age 45±17.6 yrs) had their DM fully mapped. Principal Coordinate Analysis (PCoA) of the UniFrac distance generated from taxon abundance (beta-diversity) revealed important differences in the DM profile of subjects with NAFLD when compared to controls, resulting in two distinct clusters on PCoA plots. Intriguingly, the α-diversity indexes of subjects with NAFLD were similar when compared to controls (Total number, P=0.2520; Simpson's index, P=0.8320). The DM of control subjects was characterized by high relative abundance (RA) of the Firmicutes phylum (68%), followed by Bacteroidetes, Actinobacteria and Fusobacteria (14%, 7%, and 5% respectively). Comparatively to controls, the DM of subjects with NAFLD was also characterized by high RA of the Firmicutes phylum (63%, P=0.346), but the RA of the Actinobacteria phylum was increased in these subjects (Fold change (FC)=1.60, P=0.02). Interestingly, the relative abundance of the TM7 phylum was also increased in subjects with NAFLD when compared to controls (FC=1.91, P=0.02). At order level, the RA of specific taxon from Actinobacteria and TM7 phyla were also increased in subjects with NAFLD when compared to controls, including the Bifidobacteriales order (FC=6.96, P=0.03) and CW40 order (FC=4.86, P=0.02) respectively. Additionally, the Bacillales order from Firmicutes phylum and the Enterobacteriales order from Proteobacteria, were also increased in NAFLD subjects when compared to controls.

### Example 3

KEGG L3 Metabolic pathways changes were examined. The sulfur relay system was found to be active in patients with NAFLD.

Since the small bowel microbial profile is different in subjects with NAFLD when compared to controls, especially because the abundance of the genus Actinomyces increased in NAFLD, microbial metabolic pathways also changed in the small bowel (the microbial metabolic pathway profile is highly dependable of the microbial profile). Changes in metabolic profile were related mostly with the enrichment of the sulfur-relay system pathway (highest variable importance in projection (VIP) score). This pathway facilitates several processes involving sulfur compounds (sulfur metabolism) and indicates a higher flow of this type of molecule in the small bowel. The enrichment of this pathway may reflect higher concentration of sulfur compounds in the small bowel, and can be present in the liver as well. Accordingly, the presence of sulfur granules in the GI tract is indicative of NAFLD.

### Example 4

A 30 year old patient presents with symptoms of nonalcoholic fatty liver. Penicillin is administered to the patient to inhibit, redistribute, reduce, or eradicate actinomyces in the patients GI tract.

### Example 5

A 42 year old patient presents with symptoms of non-alcoholic steatohepatitis. Prevotella is administered to the patient to inhibit, redistribute, reduce, or eradicate actinomyces in the patients GI tract.

### Example 6

A 30 year old patient presents with symptoms of nonalcoholic fatty liver. The patent is tested for levels of Actinomyces massiliensis from a duodenal aspirate sample, and the levels are found to be higher than the control level. The patient is diagnosed with nonalcoholic fatty liver. An antibiotic is administered to the patient to inhibit, redistribute, reduce, or eradicate actinomyces in the patients GI tract.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of treating nonalcoholic fatty liver disease (NAFLD) in a subject in need thereof, comprising administering to the subject an agent that inhibits or eradicates actinomyces.

2. The method of claim 1, wherein NAFLD is nonalcoholic fatty liver (NAFL).

3. The method of claim 1, wherein NAFLD is non-alcoholic steatohepatitis (NASH).

4. The method of claim 1, wherein the agent that inhibits, redistributes, reduces, or eradicates actinomyces comprises an antibiotic.

5. The method of claim 4, wherein the antibiotic is Penicillin G.

6. The method of claim 4, wherein the antibiotic is rifaximin, amoxicillin, ceftriaxone, clarithromycin, clindamycin, doxycycline, erythromycin, linezolid, meropenem, penicillin G, tazobactam, tetracycline or combinations thereof.

7. The method of claim 1, wherein the agent comprises prevotella.

8. The method of claim 1, wherein the agent is released into the gastrointestinal system of the subject.

9. The method of claim 1, wherein the agent is released into duodenum of the subject.

10. A method of diagnosing nonalcoholic fatty liver disease (NAFLD) in a subject in need thereof, comprising:
measuring a level of actinomyces in the gastrointestinal system of the subject; and
determining the presence of NALFD when a level of actinomyces is higher than a control level.

11. The method of claim 10, wherein NAFLD is nonalcoholic fatty liver (NAFL) and the level of actinomyces is higher than a control level for NALF.

12. The method of claim 10, wherein NAFLD is non-alcoholic steatohepatitis (NASH), and the level of actinomyces is higher than a control level for NASH.

13. The method of claim 10, wherein the level of actinomyces in the gastrointestinal system is measured from a biological sample selected from saliva, small intestinal aspirate, or stomach aspirate.

14. A method of diagnosing nonalcoholic fatty liver disease (NAFLD) in a subject in need thereof, comprising:
measuring a level of sulfur granules in the gastrointestinal system of the subject; and
determining the presence of NALFD when a level of sulfur granules is higher than a control level.

15. The method of claim 14, wherein NAFLD is nonalcoholic fatty liver (NAFL) and the level of actinomyces is higher than a control level for NALF.

16. The method of claim 14, wherein NAFLD is non-alcoholic steatohepatitis (NASH), and the level of actinomyces is higher than a control level for NASH.

17. The method of claim 14, wherein the level of sulfur granules is measured from a biological sample selected from saliva, small intestinal aspirate, or stomach aspirate.

18. The method of claim 14, wherein small intestinal aspirate is duodenal aspirate.
